(19) United States
(12) Patent Application Publication
(10) Pub. No.: US 2013/0126630 A1
(43) Pub. Date: May 23, 2013

(54) AIR FRESHENING SYSTEM

(71) Applicants: AMELIA H. MAJEROWSKI, Kenosha, WI (US); Mark E. Wefler, Hartsel, CO (US); Gregory A. Falduto, Grayslake, IL (US); Kevin Harrity, Oak Creek, WI (US)

(72) Inventors: AMELIA H. MAJEROWSKI, Kenosha, WI (US); Mark E. Wefler, Hartsel, CO (US); Gregory A. Falduto, Grayslake, IL (US); Kevin Harrity, Oak Creek, WI (US)

(21) Appl. No.: 13/809,512

(22) PCT Filed: Jul. 12, 2011
(86) PCT No.: PCT/US11/43766

§ 371 Date: Feb. 5, 2013

Related U.S. Application Data
(60) Provisional application No. 61/363,474, filed on Jul. 12, 2010.

(51) Int. Cl. A61L 9/12

(52) U.S. Cl. CPC A61L 9/12
USPC 239/6; 239/57

(57) ABSTRACT

An air freshening system includes a container 54 having a permeable membrane 120 disposed thereon and a volatile material 56 disposed therein. An impermeable laminate 122 is releasably attached to the permeable membrane 120 and is adapted to be removed therefrom prior to use. An attachment mechanism is adapted to retain the container on a stem 52.

### CROSS REFERENCE TO RELATED APPLICATIONS

This application claims the priority of U.S. Provisional Patent Application No. 61/363,474, filed on Jul. 12, 2010.

### REFERENCE REGARDING FEDERALLY SPONSORED RESEARCH OR DEVELOPMENT

Not applicable

### SEQUENTIAL LISTING

Not applicable

### BACKGROUND OF THE INVENTION

1. Field of the Invention

The present disclosure relates generally to an air freshening system and, more particularly, to an air freshening system having a stem and a shaped thermoformed cartridge filled with a volatile active.

2. Description of the Background of the Invention

Typical air freshening systems include scent-emanating materials located in some portion of the system and may include artificial articles, such as flowers. Frequently, a scent emitting article is disposed within a container that holds the flowers so as to hide the scent emitting article from view. In some cases, porous covers are disposed on the container to further restrict the view of the emitting article while at the same time allowing passive diffusion of the fragrant material therethrough.

In one system, a fragrant artificial flower display includes a vessel with an open top and an interior chamber defined by an inside surface and an inside surface of a bottom end. A base having a top surface is provided within the vessel and preferably conforms to the inside surface of the vessel. A stem of the flower is partially encased within the base and a gel layer is provided on top of the base within the vessel to emit a fragrance. In a different embodiment, the fragrance is provided by a rod having a fragrant gel disposed at an end thereof and placed adjacent the artificial flowers.

In a different floral system, a container holds a liquid fragrant material in a bottom portion thereof. A wick is provided in a stem of an artificial flower. The wick is adapted to rest in the liquid material such that capillary action causes the fragrant material to travel up the wick and be dispersed out of the artificial flowers.

The present disclosure contemplates an air freshening system that the consumer has complete control over. The natural look of the air freshening system provides a realistic visual display that blends into home decor, while at the same time provides the consumer unlimited options to control the strength of the fragrance being emitted from the air freshening system. Activation of the air freshening system is simple in that the consumer chooses the number of air freshening systems to activate at any given time. Further, the air freshening system is portable such that the consumer is also able to display the air freshening system in any part of the home.

### SUMMARY OF THE DISCLOSURE

According to one aspect of the present disclosure, an air freshening system includes a container having a permeable membrane disposed thereon and a volatile material disposed therein. The system also includes a stem and an attachment mechanism adapted to retain the container on the stem. An impermeable laminate is releasably attached to the permeable membrane and adapted to be removed therefrom prior to use.

In a different aspect of the present disclosure, an air freshening system includes a container holding a volatile material therein, which is attached to a stem. A permeable membrane is disposed on the container and an impermeable laminate is releasably attached to the permeable membrane, which is adapted to be removed therefrom prior to use.

In a further aspect of the present disclosure, a method for emitting a volatile material includes the step of providing a plurality of containers in the shape of a naturally occurring object. Each container includes a permeable membrane to regulate the diffusion of a volatile material disposed within the container and an impermeable laminate disposed over the permeable membrane to substantially prevent diffusion of the volatile material. Each container is attached to a flexible stem. The method also includes the step of removing at least one of the impermeable laminates from the plurality of containers at a first point in time to release a volatile material and at least one of the remaining impermeable laminates from the plurality of containers at a second point in time after the first point in time to provide a boost in the diffusion of volatile materials.

### BRIEF DESCRIPTION OF THE DRAWINGS

FIG. 1 is an isometric view of a first embodiment of an air freshening system including a plurality of stems having a thermoformed container disposed at ends thereof, wherein the air freshening system is disposed in a vase;

FIG. 2 is an isometric view of the air freshening system of FIG. 1, wherein the air freshening system is disposed in a wall-mounted unit;

FIG. 3 is an enlarged partial rear isometric view of one of the stems and thermoformed containers of FIG. 1;

FIG. 4 is a cross-sectional view of the stem and thermoformed container of FIG. 3 taken generally along the line 4-4 thereof;

FIG. 5 is a partial rear isometric view of a second embodiment of an air freshening system having a stem and a thermoformed container attached thereto;

FIG. 6 is the air freshening system of FIG. 5 with the stem removed therefrom;

FIG. 7 is a partial rear isometric view of a third embodiment of an air freshening system including a bracket attached to a thermoformed container with a stem adapted to be attached thereto;

FIG. 8 is a partial front isometric view of a fourth embodiment of an air freshening system including a first and second portion of a bracket attached by a joint and enclosing a thermoformed container with a stem adapted to be attached thereto;

FIG. 9 is an isometric view of a fifth embodiment of an air freshening system including a plurality of stems and thermoformed containers attached thereto;

FIG. 10 is an enlarged partial isometric view of the air freshening system of FIG. 9;

FIG. 11 is an isometric view of a sixth embodiment of an air freshening system, which includes a base and the thermoformed containers of FIG. 9;

FIG. 12 is a partial isometric view of a seventh embodiment of an air freshening system including a stem and a thermoformed container attached thereto by a locking mechanism;

FIG. 13 is a rear isometric view of the thermoformed container of FIG. 12;

FIG. 14 is a side elevational view of the thermoformed container of FIG. 12;

FIG. 15 is a rear isometric view of the locking mechanism of FIG. 12;

FIG. 16 is a front isometric view of the locking mechanism of FIG. 12;

FIG. 17 is another front isometric view of the locking mechanism of FIG. 12;

FIG. 18 is a front elevational view of the locking mechanism of FIG. 12; and

FIG. 19 is a front isometric view of the air freshening system of FIG. 12 with the locking mechanism in an open state.

Other aspects and advantages of the present invention will become apparent upon consideration of the following detailed description, wherein similar structures have similar reference numerals.

### DETAILED DESCRIPTION

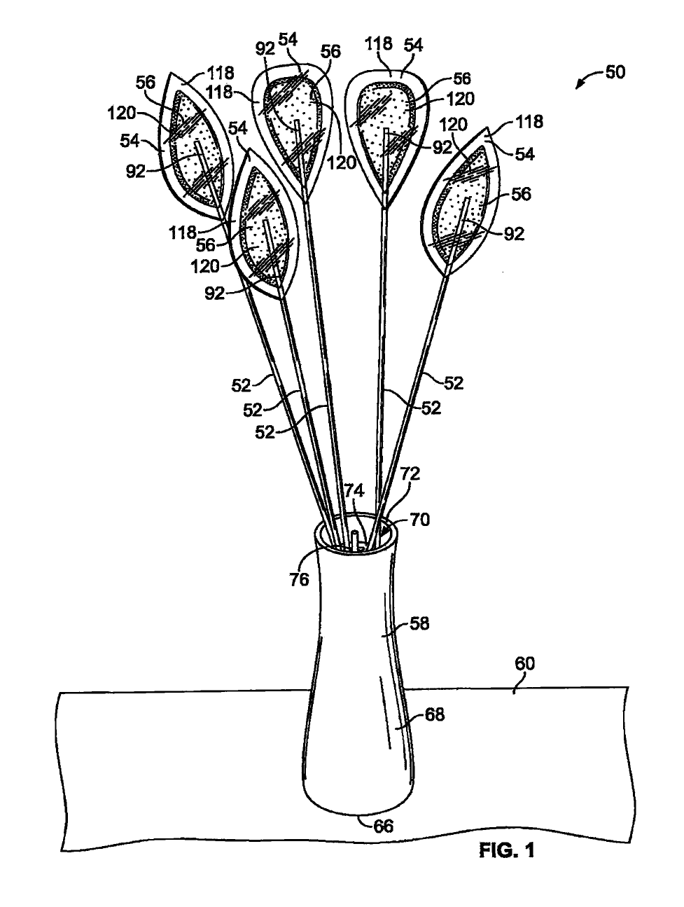

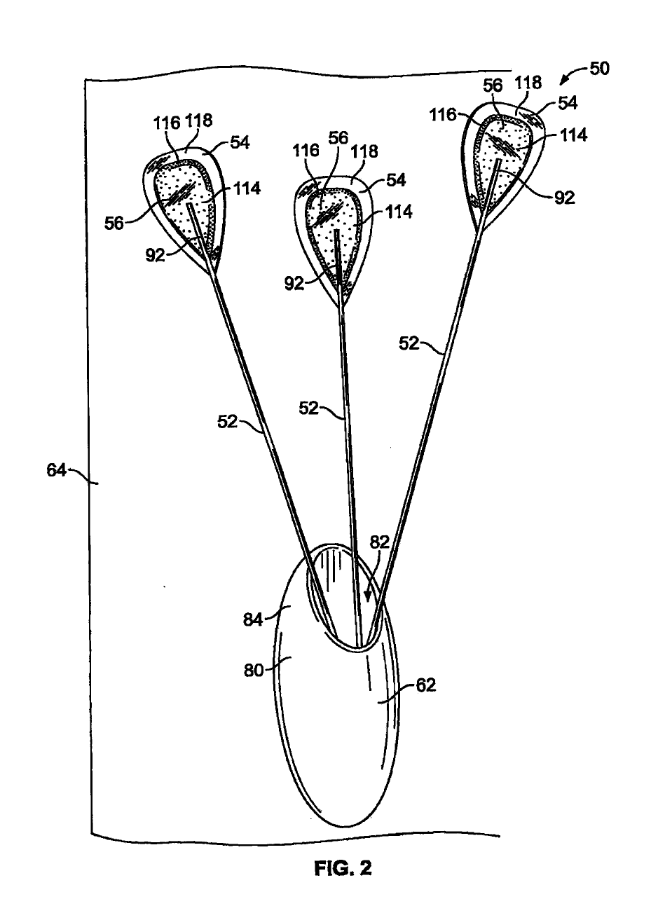

FIGS. 1 and 2 depict an air freshening system 50 comprising a stem 52 and a container 54 or cartridge in the shape of a flower attached thereto. A volatile material 56 is disposed within the thermoformed container 54 and is adapted to be diffused therefrom. The air freshening system 50 is generally adapted to be used in bunches, which comprises a plurality of stems 52 and containers 54. Any number of stems 52 and containers 54 may be used including a single stem 52 and a single container 54. As shown in FIG. 1, the air freshening system 50 is supported by a vase 58. The vase 58 depicted in FIG. 1 is adapted to rest on a substantially horizontal planar surface 60, such as a table or counter, and to hold the air freshening system 50 in an upright or substantially vertical position with respect to the planar surface 60. As shown in FIG. 2, the air freshening system 50 is depicted resting in a wall-mounted unit 62, which is adapted to interact with a wall or vertical surface 64. Although the vase 58 and the wall-mounted unit 62 are depicted, any container may be used to support and retain the air freshening system 50.

Referring to FIG. 1, the vase 58 includes a substantially flat base 66 with a curvilinear sidewall 68 extending upwardly therefrom. An orifice 70 is disposed at a top end 72 of the sidewall 68 and is adapted to allow the air freshening system 50 to be inserted therethrough. A substantially horizontal platform 74 extends across an interior of the vase 58 and includes a plurality of openings 76 therethrough adapted to support the stem(s) 52 of the air freshening system 50. In use, a consumer may insert the stem 52 into one of the plurality of openings 76. In one embodiment, a distal end (not shown) of the stem 52 rests on an interior surface of the vase 58. In another embodiment, all or a portion of the stem 52 interacts with portions of the horizontal platform 74 defining the openings 76 in an interfering relationship to hold the stem 52 upright.

As shown in FIG. 1, the thermoformed container 54 is provided in a variety of different shapes. Preferably, the shape of the thermoformed container 54 resembles a flower, an herb, a leaf, or any other botanical object. The floral and/or herbal shape of the thermoformed container 54 imparts a life-like appearance to the air freshening system 50. However, in other embodiments it is contemplated that other shapes that imitate life-like or naturally occurring objects may be imparted to the container 54.

As best seen in FIG. 2, the wall-mounted unit 62 includes a substantially oval body 80 having an opening 82 at a top end 84 thereof. Similar to the vase 58, the wall-mounted unit 62 further optionally includes a platform having openings (not shown) extending therethrough. The stern(s) 52 are adapted to be inserted into and retained in the wall-mounted unit 62 in the same way as the vase 58. Although a vase 58 and a wall-mounted unit 62 are shown, the air freshening system 50 may be retained in any other type of container and/or in no container at all. The container or vase may alternatively include a false bottom, which also may accommodate shorter length stems. Alternatively, the air freshening system 50 may include a clip (not shown) or some other mechanism that facilitates attaching the air freshening system 50 to a surface, for example, a car visor. Still further, the air freshening system 50 may be used horizontally, for example, in a drawer and/or resting adjacent the horizontal planar surface 60.

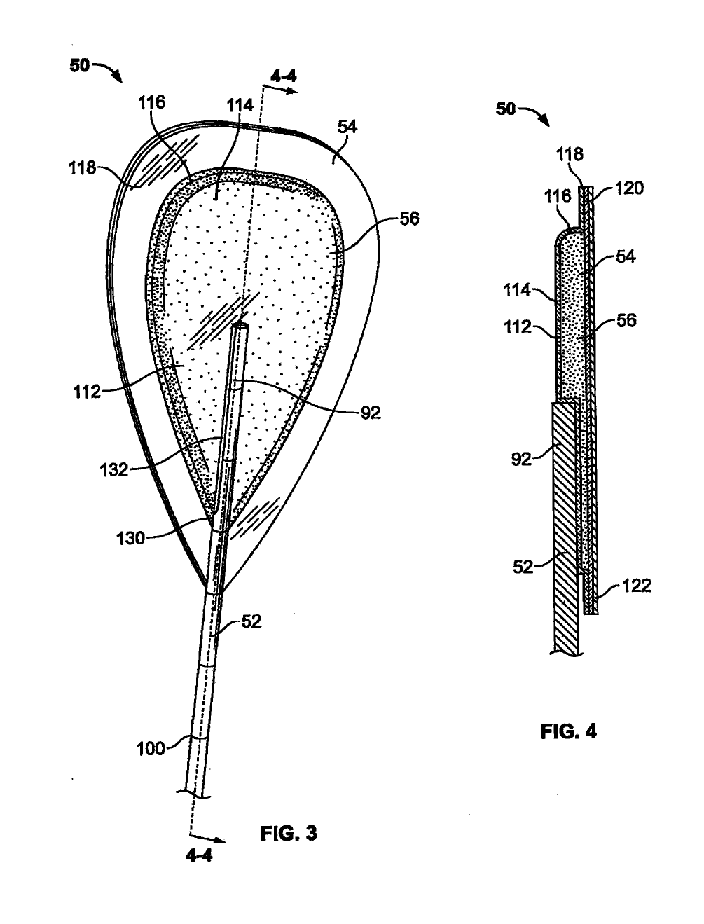

Now turning to FIG. 3, the stem 52 of the air freshening system 50 is substantially cylindrical and includes a first end (not shown) adapted to contact an interior of the vase 58 and/or wall-mounted unit 62. The stem 52 further includes a second end 92 having the thermoformed container 54 attached thereto. Although the stem 52 is depicted as cylindrical, the stem 52 may be a variety of geometric shapes and/or combinations of geometric shapes. In the present embodiment, the stem 52 has a diameter within a range of about 1 mm to about 100 mm. However, other preferred embodiments include stems 52 having a diameter within the range of about 1 mm to about 50 mm. Further, the stem 52 includes a length dimension of about 250 mm in the present embodiment. In other embodiments, the stem 52 includes a length dimension of about 100 mm to about 500 mm. In one embodiment, the diameter of the stem 52 varies along the length thereof. Illustratively, the stem 52 may have a thicker diameter at the distal end and a small diameter at various other points along the length of the stem 52 to impart flexibility and a life-like appearance. However, the length, diameter, and size characteristics of the stem 52 are readily adjustable depending on a user's needs and may be appropriately adjusted.

In one embodiment, the stem 52 is made of a wood, rattan, or bamboo material, which imparts rigid yet flexible characteristics to the stem 52. In another embodiment, the stem 52 is made of a coated wire material. Further suitable materials for the stem 52 include any natural and/or synthetic materials including, for example, polymers such as polystyrene, polyester, and polyolefin, metals, ceramics, stiffened fabrics, glass, and any other materials capable of supporting the thermoformed container 54. In one embodiment, the stem 52 is substantially solid throughout the length thereof. In a different embodiment, the stem 52 includes a hollow center and/or hollow sections throughout the length thereof.

Stems 52 may include a single continuous cylindrical shape and/or may include branches (not shown) that extend in an angular manner from the stem 52 to impart a realistic and natural appearance. The stems 52 and/or branches may be flexible such that bending is possible. In another embodiment, the stems 52 and/or branches may bend or droop with the passage of time to more realistically emulate a real plant or flower. In any of the embodiments, stems 52 having different diameters may be used in the same air freshening system to impart a more life-like appearance.

In one particular embodiment, it was preferred to provide a stem in combination with a thermoformed container that provided a relatively unchanging bend to the stem over a period of time. Using a stem with a length of about 250 mm, it was found that stems formed from polypropylene or polyethylene resulted in creep to the stem, i.e., the weight of the container on one end of the stem caused the stem to deform over time. Through experimental testing it was found that non-olefmic thermoplastics and their alloys, e.g., polystyrene, provide a relatively constant bend to the stem that does not change over time. Examples of various polystyrenes that are contemplated for use with the present embodiments include those manufactured by PolyOne, which has a place of business at 33587 Walker Road, Avon Lake, Ohio 44012, under the trade names HIPS 8 AR-M350 20110413, Impact PS 6210, Impact 6200 and 6201, Styrosun® 6600, Styron™ 498-AMST, Styron™ 487R-AMST, Styron™ 487-AMST, Styron™ 484-AMST, Styron™ 421-AMST, Styron A-Tech™ 1115-AMST, and Impact PS 9500.

As seen best in FIG. 3, the stem 52 optionally includes areas of weakness throughout the length thereof in the form of predetermined break points 100. A number of predetermined break points 100 may be provided in the stem 54 during the manufacturing process and/or before the air freshening system 50 is packaged to allow a consumer to adjust the length of the stems 54. Break points 100 are disposed along the length of the stem 52 at a number of predetermined intervals. For example, break points 100 may be spaced approximately 25 mm from each other. In use, once a consumer purchases the air freshening system 50, the consumer may take the stem(s) 52 and break off one or more portions using the break points 100. The force required to break or snap the stem 52 is less at the areas of the break points 100 than other areas of the stem 52. Alternatively, a consumer could adjust the length of the stem 52 by using a conventional cutting instrument.

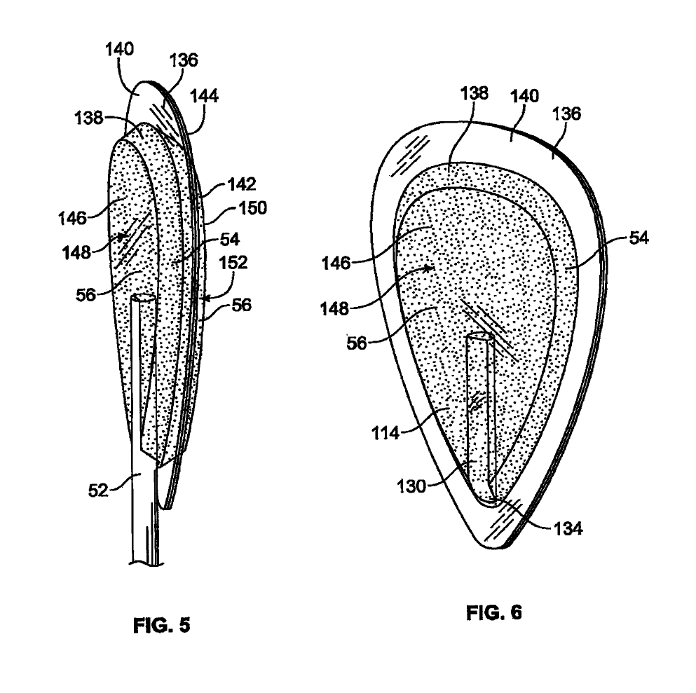

Referring to FIGS. 3 and 4, the stem 52 is adapted to interact with the thermoformed container 54 to form the air freshening system 50. Although a thermoformed container 54 is disclosed, other types of containers made by a variety of manufacturing processes are suitable for use in the air freshening system 50 including containers made of other materials. Any material may be suitable for use so long as the container is able to retain the volatile material 56. The thermoformed container 54 comprises a floral or herbal shaped thermoformed structure 112 that includes a base or bottom wall 114 and curved sidewalls 116 that extend upwardly therefrom and terminate at a peripheral flange 118. A vapor permeable membrane 120 is adhered to the peripheral flange 118 and extends across the thermoformed structure 112. The permeable membrane 120, in conjunction with the bottom wall 114 and the sidewalls 116, acts as a sealed reservoir to contain the volatile material 56. An impermeable laminate 122, shown in FIG. 4, is optionally releasably adhered to the flange 118 over the permeable membrane 120.

It is contemplated that various attachment mechanisms for securing the container 54 to the stem 52 may be utilized with the present embodiments. In one embodiment, the thermoformed container 54 includes a substantially rectangular channel 130 (see FIG. 6) disposed in the bottom wall 114 of the thermoformed structure 112. The channel 130 extends from an end 134 of the thermoformed container 54 inwardly about one third of the length of the thermoformed container 54. As shown in FIG. 4, the channel 130 is adapted to interact with the stem 52 such that the thermoformed container 54 may be securely retained on the stem 52. The channel 130 optionally includes a ledge 132 (shown in FIG. 3) that circumscribes the perimeter of the channel 130 and extends inwardly therefrom to provide a snap-fit type of interaction between the stem 52 and the thermoformed container 54. Alternatively, the channel 130 does not include a ledge, but the channel 130 is sized to the stem 52 such that an interference fit is created between the stem 52 and the thermoformed container 54 when the stem 52 is inserted into the channel 130. Still further, other securement mechanisms may be used to retain the thermoformed container 54 on the stem 52 including, for example, an adhesive and/or a tacky polymer. In still a further embodiment, the thermoformed container 54 is not connected to the stem 52 prior to use and may be used as a freestanding air freshener. In yet a different embodiment, the thermoformed container 54 does not include a channel or other groove structure that facilitates a snap-type or interference type attachment of the container 54 to the stem 52. In this embodiment, the thermoformed container 54 may be used freestanding or may be adhesively attached to the stem 52.

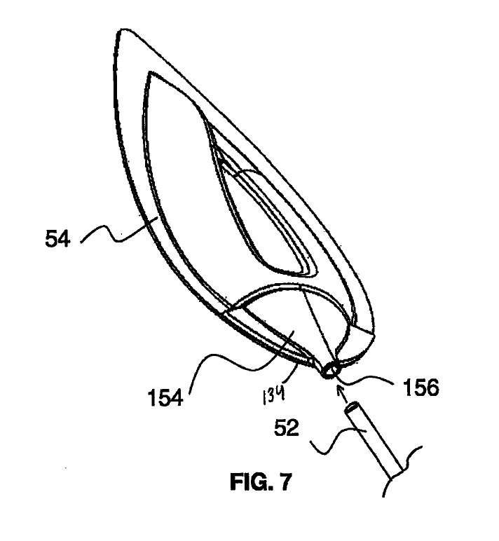

With reference to FIG. 7, a different embodiment depicts the thermoformed container 54 without the channel 130. Instead, a bracket 154 is attached to the container 54 using an ultrasonic weld. The bracket 154 is preferably disposed at the end 134 of the thermoformed container 54 and has a complementary contour so that the bracket 154 is visually appealing and blends into the thermoformed container 54. The bracket 154 further comprises an opening 156 adapted to interact with the stem 52 previously disclosed herein. The bracket 154 is preferably polyester, but may comprise other materials as well. The bracket 154 is preferably attached to the container 54 during the manufacturing process. In use, the stem 52 is inserted into the opening 156 and the thermoformed container 54 is maintained on the stem 52 via a friction fit with the bracket 154.

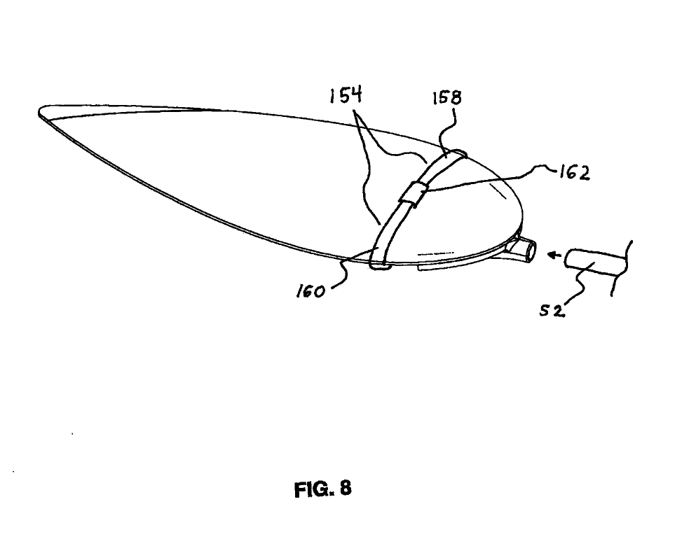

FIG. 8 depicts a different embodiment of the thermoformed container 54, which includes a bracket 154 having first and second portions 158, 160. The first and second portions 158, 160 are attached to one another at a joint 162. The joint 162 may be opened to facilitate the removal of an impermeable laminate, which will be described with greater particularity hereinbelow. In a different embodiment, the first and second portions 158, 160 are irremovably connected with one another or comprise a unitary piece that cannot be opened by a user. In this embodiment, it is preferred that the impermeable laminate be provided with one or more perforations to facilitate the removal of same by a user.

Illustratively, the thermoformed structure 112 is comprised of a recycled polyethylene terephthalate (RPET) layer adhesively bonded to a nylon laminate. The nylon laminate may also include a layer of ethylene vinyl acetate (EVA) coextruded to each side of a middle nylon layer. The nylon laminate and RPET layer of the thermoformed structure 112 in one embodiment have a thickness of about 0.3 mm to about 0.4 mm. The thermoformed structure 112 is generally in the shape of a flower with overall dimensions of about 1 mm to about 3 mm high, about 10 mm to about 100 mm long, and about 10 mm to about 70 mm wide. The sidewalls 116 and the bottom wall 114 of the thermoformed structure 112 in one embodiment are thermoformed from a single sheet of the RPET and nylon laminate that is heated, then blown and/or pressed. The thermoformed structure 112 may be clear and translucent, allowing for the visibility of the volatile material 56 therethough. In an alternative embodiment, the thermoformed structure 112 may be colored and translucent. In further embodiments, the thermoformed structure 112 may be opaque or transparent.

The flange 118 is planar and is coupled to and extends outwardly from top edges of the thermoformed structure 112. In one embodiment, the flange 118 extends outwardly from upper edges of the sidewalls 116. The flange 118 is integrally formed with the thermoformed structure 112 in, for example, a thermoforming process, as described in the previous paragraph.

Illustratively, the permeable membrane 120 has a thickness of about 0.05 mm to about 0.15 mm and has a density within a range of about 0.88 to 0.95 grams/cubic centimeter. The permeable membrane 120 may also be formed integrally with the impermeable laminate 122 and is heat fused to the flange 118 such that the permeable membrane 120 extends across the entire thermoformed structure 112. The size of the permeable membrane 120 is a key factor in the diffusion rate of the volatile material 56 therethrough. The smaller the surface area of the permeable membrane 120, the less volatile material 56 that is released, which may result in lower fragrance intensity being detected by the consumer. Other factors that may influence the diffusion rate of the volatile material include the specific composition of the volatile material, the thickness and composition of the permeable membrane, and the amount of volatile material contained within the thermoformed container.

FIG. 4 shows the permeable membrane 120 and the impermeable laminate 122 enclosing and sealing the thermoformed structure 112, thereby forming a sealed container that prevents diffusion of the volatile material 56. The container remains wholly or substantially impermeable until the user grasps a corner (not shown) of the impermeable laminate 122 and peels the impermeable laminate 122 from the permeable membrane 120, thereby exposing the permeable membrane 120 and permitting the volatile material 56 to migrate through the permeable membrane 120 and diffuse into the ambient air (FIGS. 1-3).

The permeable membrane 120 is a 5 layer co-extrusion, which includes first and second layers of a blend of low density polyethylene (LDPE) and ultra low density polyethylene (ULDPE) bonded to opposing sides of a first LDPE layer. A second LDPE layer is also bonded to the second LDPE/ULDPE layer and a polypropylene (PP) layer is bonded to the opposite side of the second LDPE layer. The permeable membrane 120 is preferably clear and translucent, allowing for visibility of the volatile material 56 therethrough.

The impermeable laminate 122 may include a layer of polypropylene, aluminum foil, and/or polyester. In one embodiment, the impermeable laminate includes a layer of aluminum foil, a layer of polypropylene (PP) adhered to one face of the aluminum foil by a PP adhesive, and a layer of polyethylene terephthalate (PET) bonded to another face of the aluminum foil. The PP layer of the foil cover is in contact with the PP layer of the vapor-permeable membrane when the foil cover is sealed thereto. Illustratively, the impermeable laminate 122 has a thickness of between about 0.1 mm and about 0.2 mm. The polyester layer is generally suitable for printing and may be the outer surface of the impermeable laminate 122.

Following placement of the volatile material 56 into the thermoformed structure 112, a seal is made between the flange 118 and the permeable membrane 120. As noted above, the impermeable laminate 122 may be attached to the flange 118 at the same time as the permeable membrane 120 if the impermeable laminate 122 and the permeable membrane 120 are co-extruded. The permeable membrane 120 and the impermeable laminate 122 may be attached to the flange 118 using any conventional means, such as an adhesive, heat sealing, and/or crimping, or the like. The seal is substantially air-tight so as to prevent leakage of air or the volatile material 56.

As noted above, the thermoformed container 54 is filled with the volatile material 56. The volatile material 56 may comprise an active ingredient for diffusion into the surrounding atmosphere, such as a fragrance, an essential oil, air freshener, odor eliminator, insecticide, and/or any other chemical components that may provide a beneficial effect or other effect. The volatile active 56 may take any form including solid, liquid, or gel. It is contemplated that any type of volatile material suited for dispersal through a permeable membrane may be used with the present embodiments described herein. The amount of volatile material 56 that is added to the thermoformed container 54 may vary depending on the desired use. Preferably, between about 0.1 grams and about 5 grams of volatile material 56 is added, more preferably about 0.5 grams to about 3 grams is added, and most preferably about 2 grams is added to the thermoformed container 54.

During a non-use state of the thermoformed container 54, the impermeable laminate 122 prevents (or substantially prevents) diffusion of the volatile material 56 through the permeable membrane 120. During an in use state, the impermeable laminate 122 is removed from the thermoformed container 54. Following removal of the impermeable laminate 122 the thermoformed container 54 transitions from a full or first condition toward an empty or second condition, which allows for the volatile material 56 to be dispersed into the atmosphere. The consumer optionally may remove any number of impermeable laminates 122 from a plurality of air dispensing systems 50 to increase fragrance dispersal as desired. For example, a consumer may wish to remove the impermeable laminates 122 immediately from two of the five air freshening systems 50 shown in FIG. 1. The consumer may thereafter remove the other three impermeable laminates 122 to provide an extra boost of fragrance dispersal a few days and/or weeks after the initial fragrance of the first two air freshening systems 50 is dispersed. One advantage to using the air freshening system(s) 50 described herein is that each air freshening system 50 is self-contained and is selectively operable by the consumer.

The thermoformed containers discussed with respect to previous embodiments include one permeable membrane thereon, however any of the thermoformed containers of the embodiments discussed herein can incorporate a plurality of permeable membranes as discussed herein. As shown in FIGS. 5 and 6, a different embodiment of the thermoformed container 54 includes a central dividing wall 136 having a first sidewall 138 extending outwardly from a first side 140 and a second sidewall 142 extending outwardly from a second side 144 of the dividing wall 136. A first permeable membrane 146 is attached to top edges of the first sidewall 138 forming an enclosed reservoir 148. A second permeable membrane 150 is attached to top edges of the second sidewall 142 forming a second enclosed reservoir 152. Alternatively, the central dividing wall 136 is preferably made of a rigid material so that the permeable membranes can be secured directly thereto. Impermeable membranes optionally cover the first and second permeable membranes 146, 150, respectively. Still further, the thermoformed container 54 may include a single permeable membrane secured to a support structure such as a dividing wall, a flange, or other rigid material.

Each reservoir 148, 152 is adapted to contain the volatile material 56 therein. In one embodiment, the volatile material 56 disposed within the first reservoir 148 is the same as the volatile material 56 in the second reservoir 152. In a different embodiment, the volatile material 56 disposed within the first reservoir 148 is different from the volatile material 56 provided in the second reservoir 152. In use, the consumer can selectively remove one or more of the impermeable membranes to release the volatile actives.

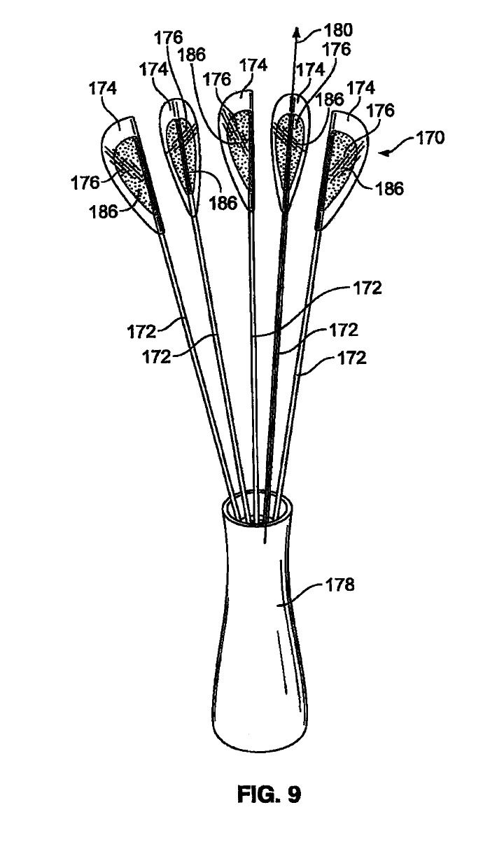

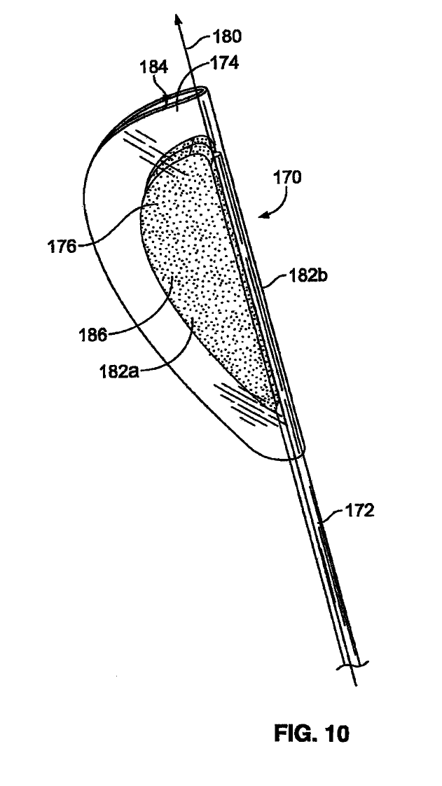

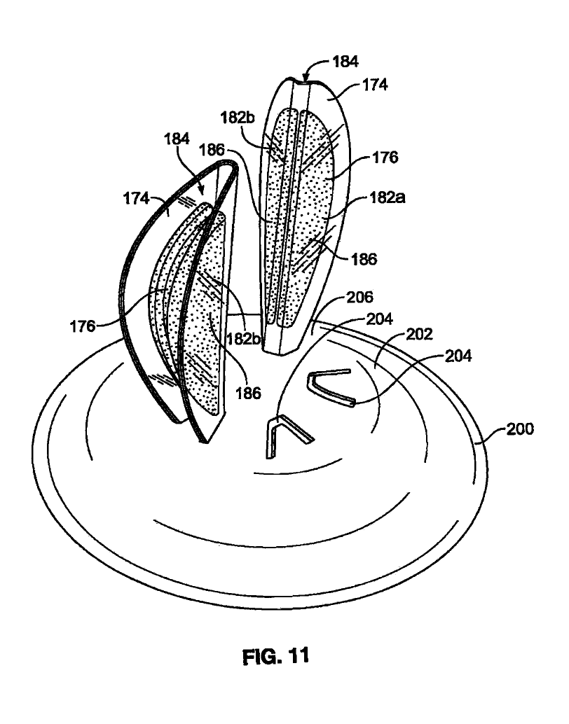

Now turning to FIGS. 9 and 10, a different embodiment of an air freshening system 170 is shown. The air freshening system 170 is similar to that of the air freshening system 50 of the first embodiment except for the differences described hereinbelow. The air freshening system 170 comprises a stem 172 and a thermoformed container 174 in the form of an artificial flower attached thereto. A volatile material 176 is disposed within the thermoformed container 174 and is adapted to be diffused therefrom. The air freshening system 170 may be supported by a vase 178 or other container. The stem 172 is similar to that of the first embodiment. The thermoformed container 174 is similar in construction to that of the thermoformed container 54, but is adapted to be folded or bent to impart a three-dimensional shape thereto.

Referring to FIGS. 10 and 11, the thermoformed container 174 is folded about a vertical axis 180 that corresponds with a longitudinal axis of the stem 172. The fold also corresponds with the longest length dimension of the thermoformed container 174. Once in a folded position, the fold of the thermoformed container 174 creates exposed sides 182a, 182b on opposite sides of the vertical axis and a central cavity 184. Preferably, a permeable membrane 186 is disposed on each of the exposed sides 182a, 182b to allow volatile material diffusion outwardly away from the central cavity 184. Although the permeable membrane 186 may be provided as one single membrane, it is also contemplated that a plurality of membranes may be included. For example, FIGS. 10 and 11 depict one permeable membrane disposed on one exposed side 182a and another permeable membrane disposed on the other exposed side 182b, forming two separate chambers. Alternatively, one container having one permeable membrane attached thereto may be folded about the axis 180 to form the v-shaped thermoformed container 174 of this embodiment. Still further, the thermoformed container 174 may include a rigid support member with at least one permeable membrane attached thereto. The container 174 may include a plurality of membranes as discussed with respect to any of the embodiments disclosed herein.

In this embodiment, the stem 172 extends upwardly and is attached to the thermoformed container 174 in the central cavity 184. The stem 172 is attached using an adhesive. Alternatively, the stem 172 and thermoformed container 174 can be attached using a channel with an undercut as described in previous embodiments.

As shown in FIG. 11, a different embodiment of the thermoformed container 174 is depicted that is adapted to be used in a base 200. The base 200 is circular and includes a domed top 202 having a plurality of substantially v-shaped cutouts 204 in a top surface 206 thereof. The base 200 can have any shape so long as it is capable of holding the thermoformed container 174. In use, a consumer may selectively place and remove thermoformed containers 174 as desired. Further, the consumer may selectively remove the impermeable laminate covers (if the containers 174 include covers) to start diffusion of the volatile material therefrom.

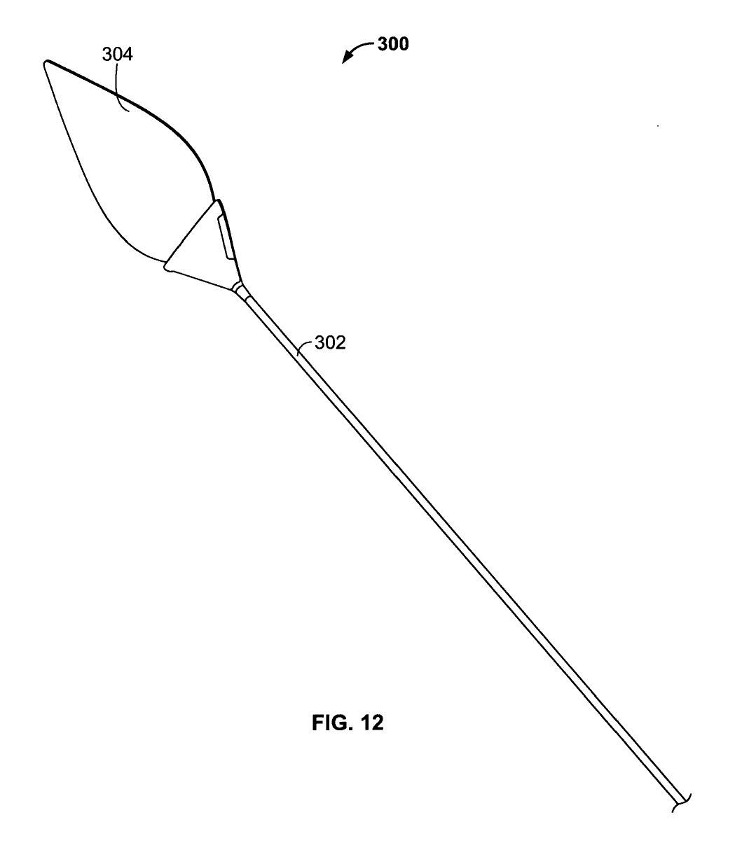

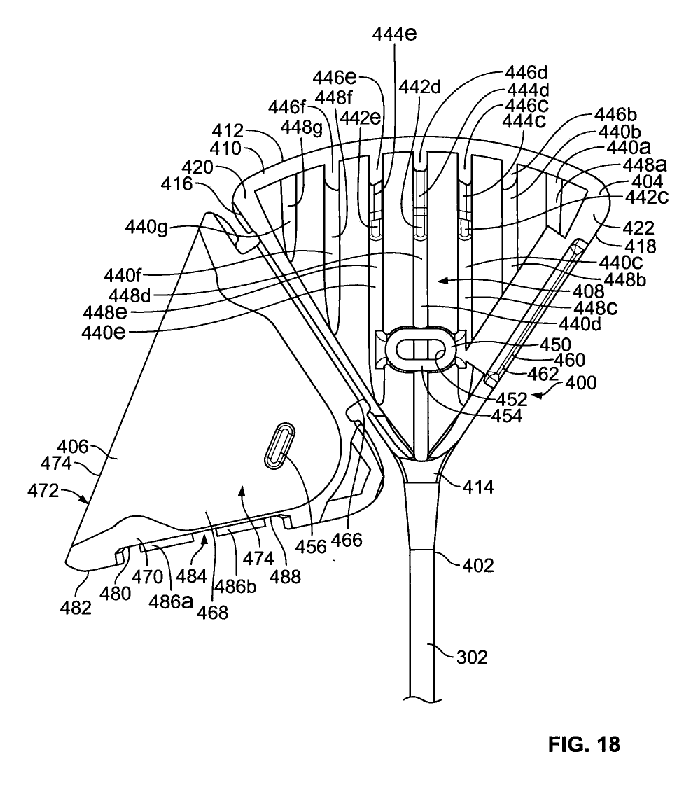

Now turning to FIGS. 12-19, an air freshening system 300 comprises a stem 302 and a container 304 or cartridge in the shape of a flower attached thereto. A volatile material is disposed within the thermoformed container 304 and is adapted to be diffused therefrom. The air freshening system 300 is similar to previous embodiments in that the materials of the stem 302 and/or the container 304 and the operation of the air freshening system 300 are substantially similar to those shown, for example, with respect to FIGS. 1-11. Although only a single stem 302 and container 304 are shown in FIGS. 12-19, any number of stems 302 and containers 304 may be utilized.

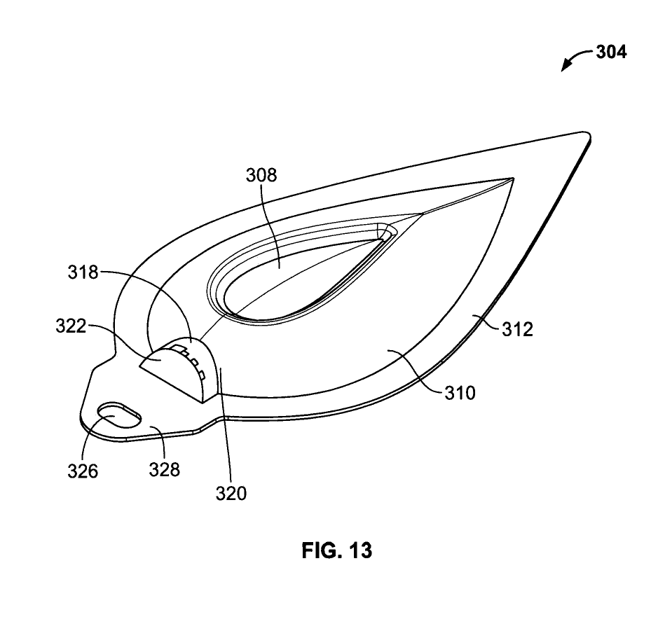

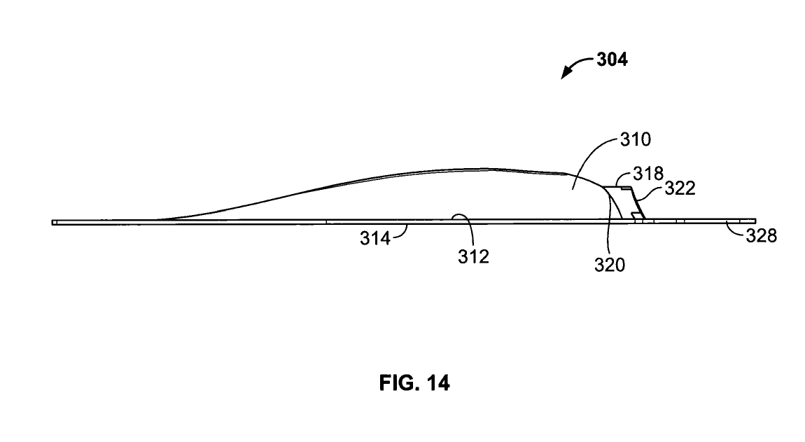

As best seen in FIGS. 13 and 14, the container 304 comprises a floral shaped thermoformed structure that includes a bottom wall 308, which in the present embodiment includes a substantially tear drop shaped depression in a central portion thereof. Bulbous sidewalls 310 extend outwardly from the bottom wall 308 and terminate at a peripheral flange 312. Other embodiments may utilize varying shapes for the bottom wall 308, which may or may not include a recess, and the sidewalls 310, to impart a visual impression of a naturally occurring object such as a leaf or floral pattern. A vapor permeable membrane 314 is adhered to the peripheral flange 312 and extends across the thermoformed structure. The permeable membrane 314, in conjunction with the bottom wall 308 and the sidewalls 310, act as a sealed reservoir to contain a volatile material. An impermeable laminate (not shown) is optionally releasably adhered to the flange 312 over the permeable membrane 314. A truncated cylindrical protrusion 318 extends from an end 320 of the sidewalls 310 and has a curved vertical endwall 322. The protrusion 318 acts as a stabilizing member when mated with the stem 302. A racetrack-shaped opening 326 is provided within a connector 328 that extends from the flange 312.

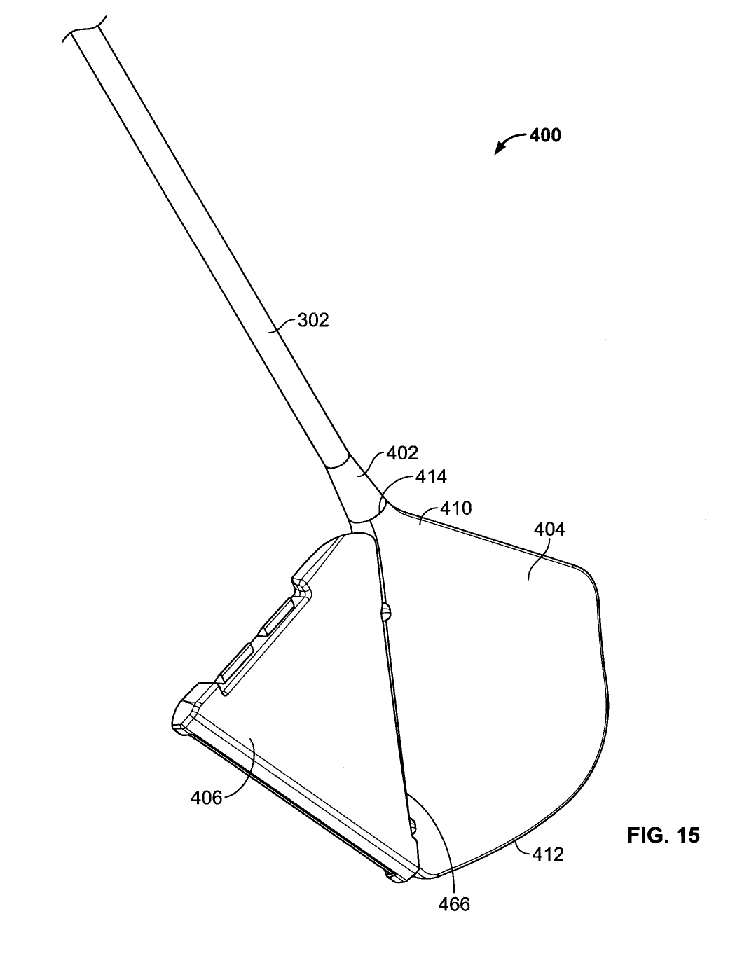

Now turning to FIGS. 15-18, the stem 302 is depicted in a first non-use state. As previously described, the stem 302 of the air freshening system 300 is substantially cylindrical and elongate. The present stem 302 includes a locking mechanism 400 disposed at a second end 402 thereof. The container 304 is adapted to be releasably locked by the locking mechanism 400 to retain the container 304 on the stem 302. Although the stem 302 is depicted as cylindrical, the stem 302 may be a variety of geometric shapes and/or combinations of geometric shapes. The length, diameter, and size characteristics of the stem 302 are readily adjustable depending on a user's needs.

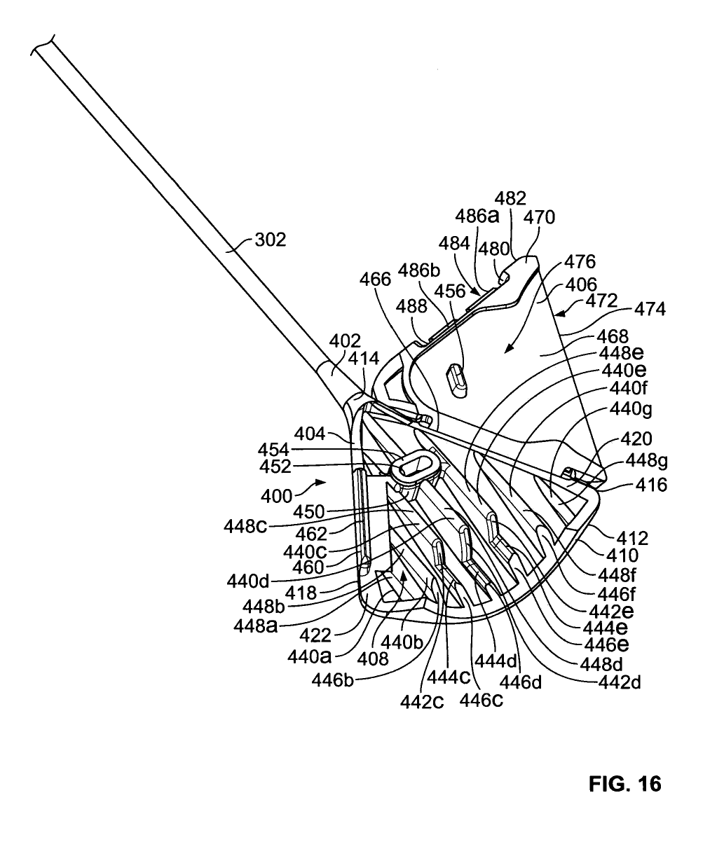

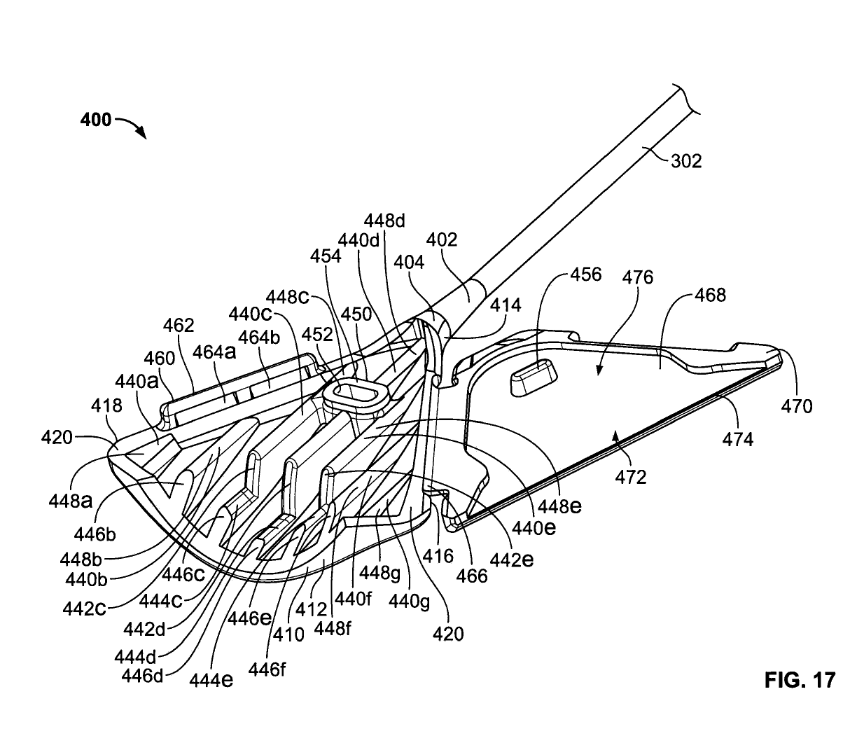

The locking mechanism 400 includes a substantially triangular base portion 404 and a similarly shaped door 406 hingedly attached thereto. The base portion 404 and door 406 form a compartment 408 that is adapted to hold and secure a portion of the container 304. In one embodiment, the locking mechanism 400 is integrally formed with the stem 302. In a different embodiment, the locking mechanism 400 is secured to the stem 302 in any manner known to those of skill in the art. The base portion 404 includes a substantially triangular curved rear wall 410 that extends from a top edge 412 downwardly toward a bottom connection point 414. As best seen in FIGS. 16 and 17, the top edge 412 varies in thickness across the width thereof. Specifically, the top edge 412 is provided with a generally concave shape between first and second edges 416, 418. Opposing first and second flat portions 420, 422 are provided adjacent the first and second edges 416, 418, respectively.

With reference to FIGS. 16-18, a plurality of elongate ribs are provided in the cavity 408 in the form of support ribs 440a-g. All of the support ribs 440a-g protrude outwardly from the rear wall 410 in a manner to provide a shaped surface for interaction with the connector 328, the protrusion 318, and/or the sidewalls 310 of the container 304. The support ribs 440a-g are generally parallel with one another and extend from portions adjacent the first and second edges 416, 418 toward the top edge 412. Support ribs 440c-e are truncated to provide clearance and support for the connector 318. The support ribs 440c-e include curved ends 442c-e, respectively, which are contoured to interact in a fitting manner with the curved vertical endwall 322 of the protrusion 318. Portions 444c-e extend from the support ribs 440c-e adjacent the curved ends 442c-e toward distal curved ends 446c-e. Portions 444c-e in combination with support ribs 440b and 440f support peripheral portions of the protrusion 318. The distal curved ends 446c-e of the support ribs 440c-e are adapted to fittingly engage the curved sidewalls 310 of the container 302. Similarly, the support ribs 440b and 440f include distal curved ends 446b and 446f, respectively, for engaging portions of the sidewall 310. The remaining upper portions 448a-g of the support ribs 440a-g, respectively, which are interior of the curved ends 442c-e, 446b, 446f, and the first and second edges 416, 418, provide a relatively planar surface for interaction with the connector 328. Upon insertion of the container 304 into the base portion 404 and closure of the door 406, which will be described in greater detail below, the compartment 408 is defined by interior portions of the door 406 and the base portion 404 to provide a complementary shape for fitting retention of the container 304.

Still referring to FIGS. 16-18, a racetrack-shaped projection 450 extends from the rear wall 410 across the support ribs 440c-e. A similarly shaped racetrack-shaped opening 452 is provided within the projection 450. An upper edge 454 of the projection 450 is substantially flush with the upper portions 448c-e of the support ribs 440c-e. The opening 452 is adapted to receive a complementary racetrack-shaped projection 456 extending from the door 406 when the locking device 400 is in a second or in-use state.

The rear wall 410 also includes a locking section 460 disposed along the second edge 418. The locking section 460 includes an upraised portion 462 that extends outwardly from the flat portion 422. Two discrete openings 464a, 464b are provided on an interior surface of the upraised portion 462 (see FIG. 17).

With reference now to FIGS. 15-18, it may be seen that the door 406 includes a hinge 466 provided along the first edge 416, which provides for a flexible attachment of the door 406 to the base portion 404. The hinge 466 may be integrally attached to one or both of the door 406 or the base portion 404. Indeed, any type of hinge may be utilized to connect the base portion 404 and the door 406 to allow for the rotatable engagement of the door 406 and the base portion 404.

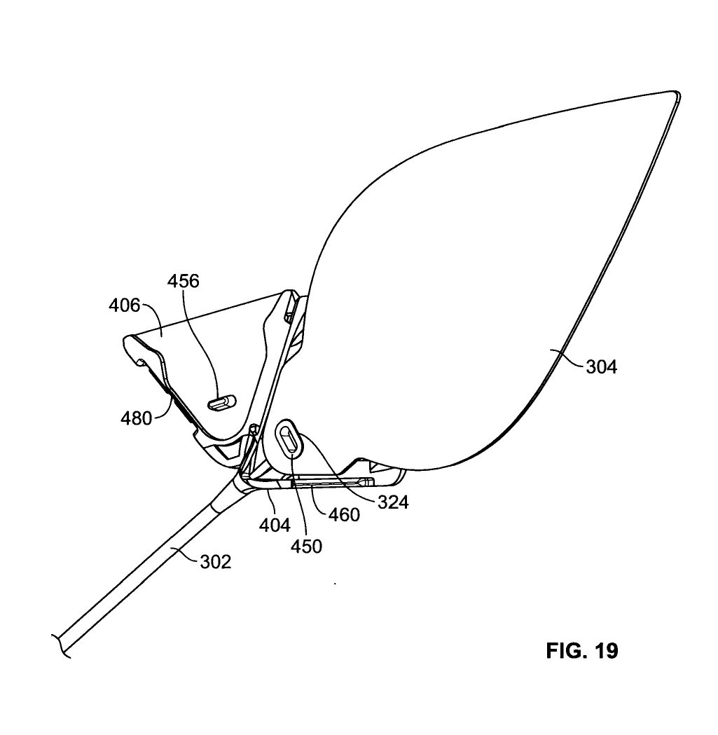

Turning to FIGS. 16-18, the door 406 is shown to include a support wall 468 and a shaped wall 470 extending upwardly therefrom. The shaped wall 470 is interrupted by an opening 472 adjacent a top edge 474 thereof The shaped wall 470 defines a space 476 complementary in shape to portions of the connector 328. The racetrack-shaped projection 456 also extends from the support wall 468 within the space 476. As previously noted, upon insertion of the container 304 into the base portion 404 and closure of the door 406, the compartment 408 provides a shaped space for fitting retainment of the connector 328, the protrusion 318, and portions of the sidewalls 310 (see FIG. 19). Further, upon placement of the container 304 into the compartment 408, the projection 450 of the base portion extends through the opening 324 of the container 304. Closure of the door 406 causes the projection 456 to be inserted into the opening 452 of the projection 450, further providing for a more secure retention of the container 304.

Retention of the door 406 in the second or in-use state is accomplished by interaction of a locking section 480 of the door 406 with the locking section 460 of the base portion 404. A sidewall 482 of the door 406 includes a rectangular recess 484. Two discrete protrusions 486a, 486b extend outwardly from a wall 488 within the recess 484. The protrusions 486a, 486b are adapted to interact with the openings 464a, 464b, respectively, within the base portion 404 when the door 406 is closed. To secure the door 406 to the base 404, a user rotates the door 406 about the hinge 466 such that the protrusions 486a, 486b ride over the upraised portion 462 and cause the protrusions 486a, 486b to be engagingly received within the openings 464a, 464b of the upraised portion 462 and the upraised portion to be seated within the recess 484. In this condition the door 406 is locked to the base portion 406. In one embodiment, the door is fixedly retained to the base portion 404 and cannot be removed without damaging the locking mechanism 400. In other embodiments, one or more of the upraised portion 462 and protrusions 486a, 486b are resilient and allow the door 406 to be opened.

Additional decorative elements may be included in the thermoformed containers discussed herein. For example, glitter, seeds, flowers, and/or the like may be included to impart a more realistic flower or herbal appearance to the air freshening system. The decorative elements can be solid materials in a variety of colors that may contrast with the air freshening system such that the decorative elements may be seen through the permeable membrane and thermoformed containers.

The fragrances and/or color schemes utilized in the air freshening system may be selected based on the shapes of the thermoformed containers. For example, a thermoformed container in the general shape of a rose may be imparted with a light red color and/or a rose-scented fragrance disposed within the thermoformed container. It is contemplated that the air freshening system can be customized by the user such that the shapes of the containers, colors of the containers and/or volatile material, and fragrance can all be selected based on user preferences for a particular theme.

Yet other decorative elements may be included in the air freshening system in the form of lighting mechanisms. For example, light-emitting diodes, fiber optic lamps, and/or other lighting sources may be included in the vases, stems, and/or containers as discussed herein. The color of the lighting mechanism may be selected to correspond to a specific herbal/floral theme. Further, the lighting may be selected to operate on a timer or light sensor such that the lighting is only active in certain settings, e.g., a low-light setting.

Further, additional elements may be used to allow the consumer to facilitate the release of the volatile active. For example, shakers (not shown) or other elements that impart movement may be used to increase the rate of diffusion of the volatile active 56. In one embodiment, shakers may be attached to the stem within the vase and/or wall-mounted unit such that the shakers are not visible during use. Other elements such as a fan, heating elements, and the like may also be used to allow the consumer to help facilitate and control the release of the volatile active(s).

Although a thermoformed container having a volatile material therein has been described throughout, other fragrancing options can be utilized in combination with and/or instead of the thermoformed containers. In one embodiment, fragrance laden beads are disposed in the vase or other support container. In a different embodiment, fragrance laden leaves are disposed along the stems and/or branches.

Further, other fragrancing devices may be used. For example, fragrancing sticks may be used. In a different embodiment, a frangible pouch may be used instead of the thermoformed container described herein such that the consumer can control the activation of the volatile material by breaking open the pouch. A still further alternative embodiment of a fragrancing device includes the use of a fragrant polymer and/or rubber that is adapted to be attached to the stem of the air freshening system. Such suitable polymers may include cellulose acetate, cellulose acetate propionate, and cellulose acetate butyrate, which are available from Rotuba, of 1401 Park Avenue South, Linden, N.J. 07036.

As apparent to those having skill in the art, packaging (not shown) may be included with the embodiments disclosed herein. The packaging preferably protects the air freshening system(s) 50, 170, the vase 58, 178 and/or the wall-mounted unit 62. The packaging may be made from any material known in the art so long as it provides a barrier that does not allow the volatile active 56 to escape therethrough.

Any of the embodiments described herein may be modified to include any of the structures or methodologies disclosed in connection with different embodiments. Further, the present disclosure is not limited to volatile material containers and/or stems of the type specifically shown.

### INDUSTRIAL APPLICABILITY

The present disclosure contemplates various air freshening systems that advantageously provide for more effective and flexible diffusion of volatiles into the atmosphere because the consumer can control the number of air freshening systems to activate. The air freshening system provides a realistic flower arrangement that blends into the decor of the home while at the same time provides control over the strength of volatiles being emitted from the flower arrangement.

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the appended claims are reserved.

We claim:

1. An air freshening system, comprising:
a container (54) having a permeable membrane (120) disposed thereon and a volatile material (56) disposed therein;
an impermeable laminate (122) releasably attached to the permeable membrane (120) and adapted to be removed therefrom prior to use;
a stem (52); and
characterized in that an attachment mechanism is adapted to retain the container (54) on the stem (52).

2. The air freshening system of claim 1, wherein the container (54) includes a base (114) and sidewalls (116) extending upwardly therefrom and a peripheral flange (118) is attached to a top edge of the sidewalls (116), and wherein at least one of the base (114) and the sidewalls (116) are in the shape of a naturally occurring object.

3. The air freshening system of claim 2, wherein the shape of the naturally occurring object is in the shape of at least one of a flower, an herb, a leaf, and a botanical object.

4. The air freshening system of claim 1, wherein the attachment mechanism includes a channel (130) for receipt of the stem (52).

5. The air freshening system of claim 4, wherein the stem (52) is retained within the channel (130) by at least one of an adhesive, a snap-fit interaction, and an interference fit.

6. The air freshening system of claim 1, wherein the attachment mechanism is a snap-fit interaction between the container (54) and the stem (52).

7. The air freshening system of claim 1, wherein the attachment mechanism is an adhesive applied to at least one of the stem (52) and the container (54).

8. The air freshening system of claim 1, wherein the stem (52) is cylindrical in cross-section and has a diameter between 1 mm and 100 mm.

9. The air freshening system of claim 8, wherein the diameter of the stem (52) varies along a length thereof between first and second ends.

10. The air freshening system of claim 1, wherein the stem comprises a polystyrene material.

11. A method for emitting a volatile material, comprising:
providing a plurality of containers (54) in the shape of a naturally occurring object, wherein each container (54) includes a permeable membrane (120) to regulate the diffusion of a volatile material (56) disposed within the container (54) and an impermeable laminate (122) disposed over the permeable membrane (120) to substantially prevent diffusion of the volatile material (56), and wherein each container (54) is attached to a flexible stem (52); and
characterized in that at least one of the impermeable laminates (122) is removed from the plurality of containers (54) at a first point in time to release a volatile material (56) and at least one of the remaining impermeable laminates (122) is removed from the plurality of containers at a second point in time after the first point in time to provide a boost in the diffusion of volatile materials (56).

12. The method of claim 11, wherein the plurality of containers (54) include a first container with a first fragrance and a second container with a second different fragrance.

13. The method of claim 11 or 12, wherein the removal of the at least one impermeable laminate (122) at the second point of time is performed upon the substantial expiration of the volatile material (56) from the at least one container having the impermeable laminate (122) removed at the first point in time.

14. The method of claim 11 or 12, wherein the removal of the at least one impermeable laminate (122) at the second point of time is performed prior to the substantial expiration of the volatile material (56) from the at least one container having the impermeable laminate (122) removed at the first point in time.

15. The method of any one of the preceding claims, wherein at least three stems (52) and containers (54) are provided.
